(19) United States
(12) Patent Application Publication
(10) Pub. No.: US 2006/0100258 A1
(43) Pub. Date: May 11, 2006

(54) Growth promotion method

(76) Inventors: Ambrogina Nicoletti, Albert Park (AU); Robert Borland, Swan Reach (AU); Thomas Graham Williams, Albert Park (AU)

Correspondence Address: HAYNES AND BOONE, LLP, 901 MAIN STREET, SUITE 3100, DALLAS, TX, 75202, US

(21) Appl. No.: 10/527,033

(22) PCT Filed: Feb. 27, 2004
(86) PCT No.: PCT/AU04/00253

§ 371 Date: Sep. 29, 2005

(30) Foreign Application Priority Data
Feb. 28, 2003 (AU) 2003900927

(51) Int. Cl. A61K 31/423; A61K 31/4184; A61K 31/36

(52) U.S. Cl. 514/375; 514/394; 514/464; 424/442

(57) ABSTRACT

The present invention relates to a method of promoting growth comprising the step of administering an effective amount of a compound of formula I: 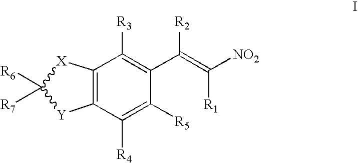 in which X and Y are either the same or different and selected from a heteroatom; [symbol]
is a double or single bond depending on the heteroatoms X and Y;
R₁ to R₅ are either the same or different and selected from hydrogen or a non-deleterious substituent; and
R₆ and R₇ are either the same or different and selected from hydrogen and a non-deleterious substituent or one of R₆ and R₇ are absent when there is a double bond present, pharmaceutically or veterinarily acceptable salts or derivatives, pro-drugs, tautomers and/or isomers thereof to a subject in need thereof.

### FIELD OF THE INVENTION

This invention relates to a growth promotion method and in particular to a method of promoting growth of an animal, more specifically intensively farmed animals, such as, pigs, cows, sheep, chickens, turkeys or fish.

### BACKGROUND

All references, including any patents or patent applications, cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents forms part of the common general knowledge in the art, in Australia or in any other country.

A popular method of livestock management is the intensive management system, which dramatically increases the population density of farmed animals and therefore improves the cost efficiency of housing, management and labour compared to less intensive management systems. However, a major consequence of intensive management is the increased opportunity for the development and spread of disease. This disease may be caused by specific pathogens introduced, for example, by errors in practice management, or by opportunistic pathogens in the contaminated and compromising intensively managed environment.

Clinical infections can lead to major disease outbreaks with high morbidity and/or mortality. Subclinical infections can interfere with the normal metabolic process of an animal. For example, interference with the gastrointestinal function of an animal can interfere with its normal eating habits. This results in poor growth and feed efficiencies by the animal, which in turn results in less efficient animal production.

Most additives can, but do not always, improve feed use and rate of live weight gain by 3 to 8 percent. However, as the additives increase the cost of a diet, their inclusion will only be cost effective if the improved performance covers the cost of the additive and the same improvement could not have been obtained by a less expensive means, such as a change in management system or an improved strain of animal.

Current feed additives approved for use in livestock diets are generally administered at 1 to 175 mg/kg feed. Many feed additives must be withdrawn from the diet before slaughter in order to minimise the presence of residues of the additive in the meat of the animal when consumed.

Current additives used to promote growth of animals include the administration of antibiotics, vitamins and minerals including copper and arsenicals, and hormone regulation therapies. Classic examples include use of the arsenicals to control swine dysentery, coccidiostats to control coccidiosis in poultry, and vitamins which compensate for vitamin deficiencies. However, feeding these molecules to animals which will be consumed by humans often raises concerns on the effect to both humans and the environment as a result of the development of antibiotic resistant bacteria and the residues remaining in the animal and in the soil. For example, there has been evidence of copper residues in pig livers and also in soils treated with pig slurry.

The development of antibiotic resistance in organisms is another major concern to consumers and compromises the ability of many widely used antibiotics to treat serious infections in humans. Not all antibiotics promote growth. For example, broad spectrum antibiotics are not generally used for this purpose. Therefore, the development of a growth promoting agent which leaves a minimal or no detectable residue is also of concern to consumers. Allergic reactions which occur as a result of consuming specific additives is known to occur. The effect of hormone supplements on consumers is of further concern and several hormone treatment programs in poultry and beef cattle have been restricted. There is increasing pressure from consumers to restrict the use of many compounds. In the European Economic Community, for example, non-authorised additives include tetracyclines, penicillins and cephalosporins, aminoglycosides, macrolides, sulfonamides and trimethoprim, nitrofurans except nitrovin, arsenicals, hormones and anti-hormones.

A requirement accordingly exists for growth promoting agents which leave minimal residues in animals to which they have been administered and in the environment.

### SUMMARY OF INVENTION

We have now found that certain substituted nitrostyrene compounds have improved growth promotant activity, require low dosage, result in low tissue levels, and have low oral toxicity.

In a first aspect, the present invention provides a method of promoting growth comprising the step of administering an effective amount of a compound of 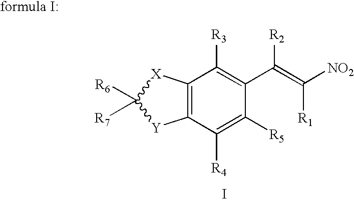
in which

X and Y are either the same or different and selected from a heteroatom;

[symbol] is a double or single bond depending on the heteroatoms X and Y;

R₁ to R₅ are either the same or different and selected from hydrogen or a non-deleterious substituent; and

R₆ and R₇ are either the same or different and selected from hydrogen and a non-deleterious substituent or one of R₆ and R₇ are absent when there is a double bond present,

pharmaceutically or veterinarily acceptable salts or derivatives, pro-drugs, tautomers and/or isomers thereof to a subject in need thereof.

The present invention also provides the use of the compound of formula I in promoting growth of a subject.

The present invention further provides the use of the compound of formula I in the manufacture of a medicament or feed for promoting growth of a subject.

The present invention still further provides the compound of formula I for use in promoting growth of a subject.

In a second aspect, the present invention provides a composition for promoting growth in a subject, which comprises the compound of formula I and a carrier.

When the composition is to be administered to a human or animal, it is preferably in the form of a pharmaceutical or veterinary composition comprising the compound of formula I and a pharmaceutically or veterinarily acceptable carrier.

Alternatively, the composition may be administered in the form of a feed for promoting growth in a subject comprising the compound of formula I.

In a third aspect, the present invention provides a growth promoting agent or a nutritional supplement comprising the compound of formula I.

The present invention also provides use of the compound of formula I as a growth promoting agent or nutritional supplement.

### DETAILED DESCRIPTION OF THE INVENTION

In the subject specification, except where the context requires otherwise due to express language or necessary implication, the words “comprise” or variations such as “comprises” or “comprising” are used in an inclusive sense, i.e. to specify the presence of the stated features but not to preclude the presence or addition of further features in various embodiments of the invention.

It must be noted that, as used in the subject specification, the singular forms “a”, “an” and “the” include plural aspects unless the context clearly dictates otherwise. Thus, for example, reference to “a compound” includes a single compound, as well as two or more compounds; and so forth.

In the compound of formula I, preferably X and Y are either the same or different and selected from O and N, more preferably both X and Y are oxygen.

Preferably R₁ and R₂ are either the same or different and selected from hydrogen, hydroxy, halogen and optionally substituted C₁₋₆ alkyl.

R₃ to R₅ are preferably either the same or different and selected from hydrogen, hydroxy, halogen, nitro, C₁₋₆ alkoxy and optionally substituted C₁₋₆ alkyl.

Preferably halogen is chlorine or bromine.

The E isomer of the compounds of formula I is preferred.

Particularly preferred are compounds of the formula I in which X, Y, [symbol] , R₆ and R₇ are as defined above; R₁ and R₂ are either the same or different and selected from hydrogen, hydroxy, Cl, Br and C₁₋₄ alkyl; and R₃ to R₅ are either the same or different and selected from hydrogen, hydroxy, Cl, Br, nitro, C₁₋₄ alkoxy and C₁₋₄ alkyl.

Specific examples of the compounds of the present invention are as follows:
(1) X and Y are O, R₁ is methyl and R₂ to R₇ are hydrogen (3,4-methylenedioxy-β-methyl-β-nitrostyrene) (hereinafter referred to as “Iksin”) 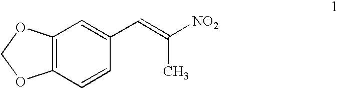
(2) X and Y are O and R₁ to R₇ are hydrogen (3,4-methylenedioxy-β-nitrostyrene) 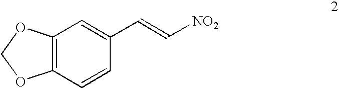
(3) X is N, Y is NH, R₁ is methyl, R₂ to R₆ are hydrogen and R₇ is absent (benzimidazole-5-β-nitropropylene) 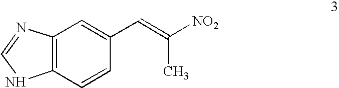
(4) X is N, Y is NH, R₁ to R₅ are hydrogen, R₆ is methyl and R₇ is absent (2-methyl benzimidazole-5-β-nitroethylene) 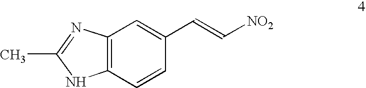
(5) X is O, Y is N, R₁ to R₆ are hydrogen and R₇ is absent (benzoxazole-5-β-nitroethylene) 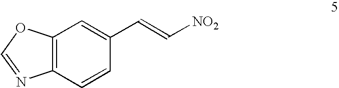
(6) X is N, Y is O, R₁ is methyl, R₂ to R₅ are hydrogen, R₆ is methyl and R₇ is absent (2-methyl benzoxazole-5-β-nitropropylene)

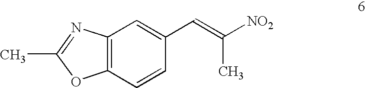

By “pharmaceutically acceptable derivative” is meant any pharmaceutically acceptable salt, hydrate, ester, amide, active metabolite, analogue, residue or any other compound which is not biologically or otherwise undesirable and induces the desired pharmacological and/or physiological effect.

The salts of the compound of formula I are preferably pharmaceutically acceptable, but it will be appreciated that non-pharmaceutically acceptable salts also fall within the scope of the present invention, since these are useful as intermediates in the preparation of pharmaceutically acceptable salts. Examples of pharmaceutically acceptable salts include salts of pharmaceutically acceptable cations such as sodium, potassium, lithium, calcium, magnesium, ammonium and alkylammonium; acid addition salts of pharmaceutically acceptable inorganic acids such as hydrochloric, orthophosphoric, sulphuric, phosphoric, nitric, carbonic, boric, sulfamic and hydrobromic acids; or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulphonic, trihalomethanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

In addition, some of the compounds of the present invention may form solvates with water or common organic solvents. Such solvates are encompassed within the scope of the invention.

The term “pro-drug” is used herein in its broadest sense to include functional derivatives of the compound of formula I which are readily convertible in vivo to the compound of formula I. Conventional procedures for the selection and preparation of suitable prodrug derivatives are described, for example, in “Design of Prodrugs” ed. H. Bundgaard, Elsevier, 1985.

The term “tautomer” is used herein in its broadest sense to include compounds of formula I which are capable of existing in a state of equilibrium between two isomeric forms. Such compounds may differ in the bond connecting two atoms or groups and the position of these atoms or groups in the compound.

The term “isomer” is used herein in its broadest sense and includes structural, geometric and stereo isomers. As the compound of formula I may have one or more chiral centres, it is capable of existing in enantiomeric forms.

The term “heteroatom” denotes O, N or S.

The term “non-deleterious substituent” is used herein in its broadest sense and refers to a substituent which does not have a deleterious effect on the growth promoting property of the compound. Examples include alkyl, alkenyl, alkynyl, aryl, halo, haloalkyl, haloalkenyl, haloalkynyl, haloaryl, hydroxy, alkoxy, alkenyloxy, aryloxy, benzyloxy, haloalkoxy, haloalkenyloxy, haloaryloxy, nitro, nitroalkyl, nitroalkenyl, nitroalkynyl, nitroaryl, nitroheterocyclyl, amino, alkylamino, dialkylamino, alkenylamino, alkynylamino, arylamino, diarylamino, benzylamino, dibenzylamino, acyl, alkenylacyl, alkynylacyl, arylacyl, acylamino, diacylamino, acyloxy, alkylsulphonyloxy, arylsulphenyloxy, heterocyclyl, heterocycloxy, heterocyclamino, haloheterocyclyl, alkylsulphenyl, arylsulphenyl, carboalkoxy, carboaryloxy mercapto, alkylthio, arylthio, acylthio and phosphorus-containing compounds.

Particularly suitable non-deleterious substituents are alkyl, alkenyl, alkynyl, halo, haloalkyl, haloalkenyl, haloalkynyl, hydroxy, alkoxy, alkenyloxy, haloalkoxy, haloalkenyloxy, nitro, nitroalkyl, nitroalkenyl and nitroalkynyl.

In a preferred embodiment the non-deleterious substituents are C₁₋₆ alkyl, halo, hydroxy, C₁₋₆ alkoxy and nitro.

The term “halogen” refers to fluorine, chlorine, bromine and iodine, preferably chlorine and bromine.

The term “alkoxy” is used herein in its broadest sense and refers to straight chain, branched chain or cyclic oxy-containing radicals each having alkyl portions, preferably C₁₋₆ alkyl, more preferably C₁₋₄ alkyl. Examples of such alkoxy groups are methoxy, ethoxy, propoxy, butoxy and t-butoxy.

The terms “C₁₋₄ alkyl” or “C₁₋₆ alkyl” used either alone or in compound words such as “optionally substituted C₁₋₄ or C₁₋₆ alkyl” refer to straight chain, branched chain or cyclic hydrocarbon groups having from 1 to 6 carbon atoms. Illustrative of such alkyl groups are methyl, ethyl, propyl, isopropyl, butyl, isobutyl, sec-butyl, tert-butyl, pentyl, neopentyl, hexyl, cyclopropyl, cyclobutyl, cyclopentyl or cyclohexyl.

The term “subject” as used herein refers to any subject in which the promotion of growth is desired. The subject may have or be at risk of having a disease or condition which requires growth promotion. The subject may be an animal or a human. While it is particularly contemplated that the compounds of the invention are suitable for treatment of domestic animals such as horses, ponies, donkeys, mules, llama, alpaca, pigs, cattle, sheep, birds and fish, or zoo animals such as primates, felids, canids, bovids, and ungulates, they are also applicable to use in humans and companion animals such as dogs and cats.

Preferably the animal is a mammal, bird or fish.

Suitable mammals include members of the orders Primates, Rodentia, Lagomorpha, Cetacea, Carnivora, Perissodactyla and Artiodactyla. Members of the orders Perissodactyla and Artiodactyla are particularly preferred because of their economic importance.

For example, Artiodactyla comprises approximately 150 living species distributed through nine families: pigs (Suidae), peccaries (Tayassuidae), hippopotamuses (Hippopotamidae), camels (Camelidae), chevrotains (Tragulidae), giraffes and okapi (Giraffidae), deer (Cervidae), pronghorn (Antilocapridae), and cattle, sheep, goats and antelope (Bovidae). Many of these animals, such as goats, sheep, cattle and pigs have very similar biology and share high degrees of genomic homology.

The order Perissodactyla comprises horses and donkeys, which are both economically important and closely related. Indeed, it is well known that horses and donkeys interbreed.

Preferably the mammal is a pig, cow or sheep.

Examples of birds include members of the order Anseriformes, which includes geese and ducks; Galliformes, which includes chickens, turkeys, quail, pheasants, guinea fowl and pea fowl; and Columbiformes, which includes pigeons and doves. Preferably the bird is a chicken, turkey, duck or goose.

Examples of fish include Clupeiforms, Perciformes, Gadiformes, Pleuronectiformes, Cypriniformes, Crustaceans and Molluscs.

In a preferred embodiment, the subject is an animal, more preferably an intensively farmed animal such as mammals, for example, pigs, cows or sheep; birds, for example, chickens such as broiler chickens, turkeys, ducks or geese; or fish.

As used herein, the term “farmed intensively” means farmed with the aim of achieving maximum production within a limited area.

Without wishing to be bound be theory, it is believed that compounds of formula I act to promote the growth of a subject by inhibiting the growth of pathogens in the gastrointestinal tract as well as having a direct effect of feed utilisation.

The promotion of growth may be determined by any suitable known method. In a preferred method, promotion of growth is determined by an increase in the weight, length, and/or height or a decrease in food intake, a reduction in time to reach marketable weight and/or instance of microbial infection of the subject as compared to a control subject. In a more preferred method, promotion of growth is determined by an increase in the weight of an subject as compared to a control subject.

It will be clearly understood that the terms “promotion of growth” and “promoting growth” mean the increase of growth of an subject when compared to a control subject.

As used herein, the term “control subject” means a subject of the same species, age and sex as the subject whose growth is being promoted however, the control subject has not been administered the compound of formula

As used herein, the term “effective amount” is meant an amount of a compound of the present invention effective to yield the desired growth promoting activity.

The specific “effective amount” will, obviously, vary with such factors as the particular condition being treated, the physical condition of the subject, the type of subject being treated, the duration of the treatment, the nature of concurrent therapy (if any), the specific formulations employed and the structure of the compound or its derivatives.

Dosage levels of the compound of formula I of the present invention may be of the order of up to about 1 g per kilogram body weight. The amount of active ingredient that may be combined with the carrier materials to produce a single dosage will vary depending upon the animal treated and the particular mode of administration. For example, a formulation intended for oral administration to humans may contain up to about 1 g of an active compound with an appropriate and convenient amount of carrier material which may vary from about 5 to about 95 percent of the total composition. Dosage unit forms will generally contain between from about 5 mg to about 500 mg of active ingredient. When used in a feed, the amount of active ingredient will be about 10 ppm to about 100 ppm.

Optionally the compounds of the invention are administered in a divided dose schedule, such that there are at least two administrations in total in the schedule. Administrations are given preferably at least every two hours for up to four hours or longer; for example the compound may be administered every hour or every half hour. In one preferred embodiment, the divided-dose regimen comprises a second administration of the compound of the invention after an interval from the first administration sufficiently long that the level of active compound in the blood has decreased to approximately from 5-30% of the maximum plasma level reached after the first administration, so as to maintain an effective content of active agent in the blood. Optionally one or more subsequent administrations may be given at a corresponding interval from each preceding administration, preferably when the plasma level has decreased to approximately from 10-50% of the immediately-preceding maximum.

The compounds of the present invention may additionally be combined with other molecules to provide an operative combination. It is intended to include any chemically compatible combination of pharmaceutically- or veterinarily-active agents, as long as the combination does not eliminate the activity of the compound of formula I. It will be appreciated that the compound of the invention and the other molecule(s) may be administered separately, sequentially or simultaneously.

Other molecules which may be used to promote growth include other antimicrobials in reduced amounts, vitamins and/or minerals.

As used herein, a “pharmaceutical carrier” is a pharmaceutically acceptable solvent, suspending agent or vehicle for delivering the compound of formula I to a subject. The carrier may be liquid or solid and is selected with the planned manner of administration in mind. Each carrier must be pharmaceutically or veterinarily “acceptable” in the sense of being compatible with other ingredients of the composition and non-injurious to a subject.

The pharmaceutically or veterinarily acceptable carrier is preferably an organic solvent such as acetone, benzene, acetonitrile, DMSO or an alcohol, for example, methanol or ethanol. While the compounds of formula I show a poor solubility in water, when water is combined with an organic solvent a stable mixture is formed.

Carriers suitable for use in the preparation of the feed supplements include alfalfa meal, soybean meal, cottonseed oil meal, linseed oil meal, sodium chloride, corn meal, cane molasses, urea, bone meal, fish meal, corncob meal, calcium chloride, vegetable or plant oils such as olive oil or canola oil and other similar materials. Use of the carriers in feed supplements promote uniformity of distribution of the compound of formula I in the finished feed into which the supplement is blended. It thus performs an important function by ensuring proper distribution of the compound of formula I throughout the feed.

It will be understood that the compound of formula I can be administered by any means. Preferably, the compound is administered orally, topically, or parentally, more preferably orally, most preferably orally in the form of a feed. The compound of formula I may be administered orally, topically, or parenterally in dosage unit formulations containing conventional non-toxic pharmaceutically or veterinarily acceptable carriers, adjuvants, and vehicles. The term parenteral as used herein includes subcutaneous injections, aerosol for administration to lungs or nasal cavity, intravenous, intramuscular, intrathecal, intracranial, injection or infusion techniques.

The present invention also provides suitable topical, oral and parenteral pharmaceutical or veterinary formulations for use in the methods of the present invention. The compounds of the present invention may be administered orally as a feed, or as tablets, aqueous or oily suspensions, lozenges, troches, powders, granules, emulsions, capsules, syrups or elixirs. As used herein, the word “feed” means food or drink.

The composition for oral use may contain one or more agents selected from the group of sweetening agents, flavouring agents, colouring agents and preserving agents in order to produce pharmaceutically or veterinarily elegant and palatable preparations. Suitable sweeteners include sucrose, lactose, glucose, aspartame or saccharin. Suitable disintegrating agents include corn starch, methylcellulose, polyvinylpyrrolidone, xanthan gum, bentonite, alginic acid or agar. Suitable flavouring agents include peppermint oil, oil of wintergreen, cherry, orange or raspberry flavouring. Suitable preservatives include sodium benzoate, vitamin E, alphatocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate. The tablets contain the active ingredient in admixture with non-toxic pharmaceutically or veterinarily acceptable excipients which are suitable for the manufacture of tablets.

These excipients may be, for example, (1) inert diluents, such as calcium carbonate, lactose, calcium phosphate or sodium phosphate; (2) granulating and disintegrating agents, such as corn starch or alginic acid; (3) binding agents, such as starch, gelatin or acacia; and (4) lubricating agents, such as magnesium stearate, stearic acid or talc. These tablets may be uncoated or coated by known techniques to delay disintegration and absorption in the gastrointestinal tract and thereby provide a sustained action over a longer period. For example, a time delay material such as glyceryl monostearate or glyceryl distearate may be employed. Coating may also be performed using techniques described in the U.S. Pat. Nos. 4,256,108; 4,160,452; and 4,265,874 to form osmotic therapeutic tablets for control release.

The compound of formula I as well as the pharmaceutically or veterinarily active agent useful in the method of the invention can be administered, for in vivo application, parenterally by injection or by gradual perfusion over time independently or together. Administration may be intravenously, intraarterial, intraperitoneally, intramuscularly, subcutaneously, intracavity, transdermally or infusion by, for example, osmotic pump. For in vitro studies the agents may be added or dissolved in an appropriate biologically acceptable solvent or buffer and added to a cell or tissue.

Preparations for parenteral administration include sterile aqueous or non-aqueous solutions, suspensions, and emulsions. Examples of non-aqueous solvents are propylene glycol, polyethylene glycol, vegetable or plant oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's intravenous vehicles include fluid and nutrient replenishers, electrolyte replenishers (such as those based on Ringer's dextrose), and the like. Preservatives and other additives may also be present such as, for example, other antimicrobials, anti-oxidants, chelating agents, growth factors and inert gases and the like.

Generally, the terms “treating”, “treatment” and the like are used herein to mean affecting a subject, tissue or cell to obtain a desired pharmacologic and/or physiologic effect. The effect may be prophylactic in terms of completely or partially preventing a disease or sign or symptom thereof, and/or may be therapeutic in terms of a partial or complete cure of a disease. “Treating” as used herein covers any treatment of, or prevention of disease in a subject, and includes: (a) preventing the disease from occurring in a subject that may be predisposed to the disease, but has not yet been diagnosed as having it; (b) inhibiting the disease, i.e., arresting its development; or (c) relieving or ameliorating the effects of the disease, i.e., cause regression of the effects of the disease.

The invention includes various pharmaceutical or veterinary compositions useful for ameliorating disease. The pharmaceutical or veterinary compositions according to one embodiment of the invention are prepared by bringing the compound of formula I, analogues, derivatives or salts thereof, or combinations of the compound of formula I and one or more pharmaceutically or veterinarily active agents into a form suitable for administration to a subject using carriers, excipients and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial agents, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 20th ed. Williams & Williams (2000), the British National Formulary, 43^{rd} edition (British Medical Association and Royal Pharmaceutical Society of Great Britain, 2000), the contents of which are hereby incorporated by reference. The pH and exact concentration of the various components of the pharmaceutical or veterinary composition are adjusted according to routine skills in the art. See Goodman and Gilman's The Pharmacological Basis for Therapeutics (7th ed., 1985).

The pharmaceutical or veterinary compositions are preferably prepared and administered in dose units. Solid dose units may be tablets, capsules and suppositories. For treatment of a subject, depending on activity of the compound, manner of administration, nature and severity of the disorder, age and body weight of the animal, different daily doses can be used. Under certain circumstances, however, higher or lower daily doses may be appropriate. The administration of the daily dose can be carried out both by single administration in the form of an individual dose unit or else several smaller dose units and also by multiple administration of subdivided doses at specific intervals.

The pharmaceutical or veterinary compositions according to the invention may be administered locally or systemically in a therapeutically effective dose. Amounts effective for this use will, of course, depend on the severity of the disease and the weight and general state of the subject. Typically, dosages used in vitro may provide useful guidance in the amounts useful for in situ administration of the pharmaceutical or veterinary composition, and animal models may be used to determine effective dosages for the promotion of growth. Various considerations are described, e.g., in Langer, Science, 249: 1527, (1990). Formulations for oral use may be in the form of hard gelatin capsules wherein the active ingredient is mixed with an inert solid diluent, for example, calcium carbonate, calcium phosphate or kaolin. They may also be in the form of soft gelatin capsules wherein the active ingredient is mixed with water or an oil medium, such as peanut oil, liquid paraffin or olive oil.

Aqueous suspensions normally contain the active materials in admixture with excipients suitable for the manufacture of aqueous suspension. Such excipients may be (1) suspending agent such as sodium carboxymethyl cellulose, methyl cellulose, hydroxypropylmethylcellulose, sodium alginate, polyvinylpyrrolidone, gum tragacanth and gum acacia; (2) dispersing or wetting agents which may be (a) naturally occurring phosphatide such as lecithin; (b) a condensation product of an alkylene oxide with a fatty acid, for example, polyoxyethylene stearate; (c) a condensation product of ethylene oxide with a long chain aliphatic alcohol, for example, heptadecaethylenoxycetanol; (d) a condensation product of ethylene oxide with a partial ester derived from a fatty acid and hexitol such as polyoxyethylene sorbitol monooleate, or (e) a condensation product of ethylene oxide with a partial ester derived from fatty acids and hexitol anhydrides, for example polyoxyethylene sorbitan monooleate.

The pharmaceutical or veterinary compositions may be in the form of a sterile injectable aqueous or oleagenous suspension. This suspension may be formulated according to known methods using those suitable dispersing or wetting agents and suspending agents which have been mentioned above. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono-or diglycerides. In addition, fatty acids such as oleic acid find use in the preparation of injectables.

The compounds of formula I may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

The compounds of formula I may preferably be presented for use in the form of veterinary compositions, which may be prepared, for example, by methods that are conventional in the art. Examples of such veterinary compositions include those adapted for: oral administration, external application, for example drenches (e.g. aqueous or non-aqueous solutions or suspensions); tablets or boluses; powders, granules or pellets for admixture with feed stuffs; pastes for application to the tongue; parenteral administration for example by subcutaneous, intramuscular or intravenous injection, e.g. as a sterile solution or suspension; or (when appropriate) by intramammary injection where a suspension or solution is introduced in the udder via the teat; topical applications, e.g. as a cream, ointment or spray applied to the skin; or intravaginally, e.g. as a pessary, cream or foam.

### BRIEF DESCRIPTION OF THE DRAWINGS

In the examples, reference will be made to the accompanying drawings in which:

FIG. 1 is a graph showing plasma levels of Iksin in chicks after treatment with Iksin at 100 μg/g in DMSO and at 400 μg/g in distilled water/Tween20; and

FIG. 2 is a graph showing the average percentage weight increase in chicks after single dosing with Iksin at 12 μg/g in DMSO, 100 μg/g in DMSO, 200 μg/g in distilled water/Tween20 and 400 μg/g in distilled water/tween20 compared to untreated chicks.

### EXAMPLES

The invention will now be described in detail by way of reference only to the following non-limiting examples.

### Example 1

### Determination of Dose Range

Day-old chicks were orally administered Iksin in DMSO at 100 μg/g, 125 μg/g, 150 μg/g and 200 μg/g body weight and in Distilled Water (DW)/5% Tween-20 at 100 μg/g, 200 μg/g, 400 μg/g and 500 μg/g body weight.

The maximum tolerated oral dose of Iksin for day-old broiler chicks was 200 μg/g Iksin/DMSO and ≧500 μg/g in DW/T20.

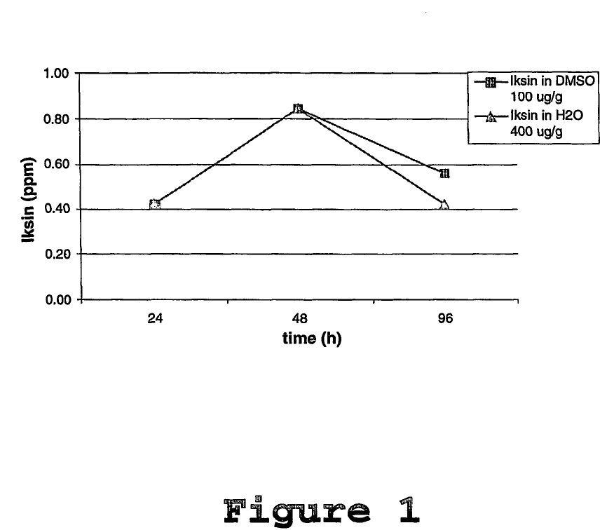

Very little Iksin was absorbed from a single oral dosing. Iksin blood levels for doses of 12 μg/g DMSO or 200 μg/g DW were below the limits of detection. At 100 μg/g DMSO and 400 μg/g DW peak levels of 850 μg/g were observed at 48 h (FIG. 1).

Effect on Chick Growth

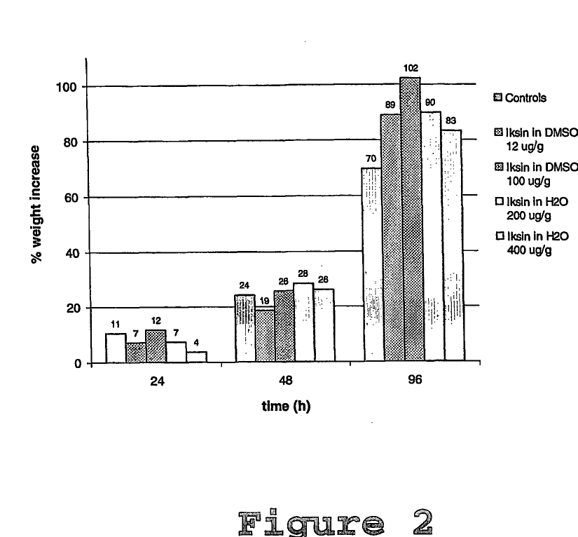

Treatment with all doses and formulations of Iksin resulted in increased weight gain in chicks 4 days after a single treatment (FIG. 2). Chicks treated with Iksin 100 μg/g DMSO showed a 32% increase in weight gain over the group of control chicks. Treatment with Iksin 12 μg/g DMSO, 200 and 400 μg/g DW/T20 gave a mean weight gain of 17%.

Conclusions

Oral administration of Iksin, formulated both in water and DMSO, is effective in promoting chick growth and is poorly absorbed with low tissue residues. It is anticipated that the results observed in the chicks will also be observed in other animals and humans.

### Example 2

### Repeat Poultry Trial

Objective

To investigate the potential of Iksin as a performance enhancing feed additive for broiler chickens and to determine some safety criteria for its use.

Purpose of Trial

The purpose of this trial is to compare the performance of Iksin supplemented feed against negative control feed (no additive) and positive control feed (Zn bacitracin).

The parameters to be measured are

(i) weight gain and feed conversion rates at weekly intervals;

(ii) pathogen colonisation in the gastrointestinal tract with Salmonella and Campylobacter by cloacal swab during rearing and by caecum contents at time of processing;

(iii) the residue of Iksin in breast muscle tissue, fat and liver at time of processing and after 5 days withdrawal; and

(iv) the level of residues in faeces and broiler litter at time of processing and after 5 days withdrawal.

Trial Protocol and Methods

Manufacture of Rations

Standard no-additive rations of Broiler Starter and Grower-Finisher Feeds (Ridley) supplemented with Iksin in canola oil and Zn bacitracin (commercial Albac 150) at the concentration recommended by the manufacturer will be prepared by Longerenong Agricultural College.

Chickens

720 1-day old commercial broiler chicks from Hazeldene Bendigo.

Treatment Groups

Three treatment groups (240 birds each) with 4 replicates (60 birds each)
1 rations supplemented with Iksin at 50 mg/kg
2 rations supplemented with Zn bacitracin
3 rations with no additives
OR
Six treatment groups of 2 replicates each (or 3 replicates +1)

1a Iksin 50 mg/kg+old litter
1b Iksin 50+new litter
2a Zn bacitracin+old litter
2b Zn bacitracin+new litter
3a No additives+old litter
3b No additives+new litter
   Broiler Chick Management

Birds will be housed according to the industry standard for commercial broiler flocks spatial requirements (0.67 sq ft/bird) and environmental conditions.

Each replicate will be separately housed with separate feed and water supplies.

Feed and water will be available ad libitum.

Pens will be seeded with broiler litter (16 weeks old) from a commercial rearing shed at Longerenong.

This litter will be assayed for presence of Clostridium perfringens, Salmonella enterica and Campylobacter spp to confirm contamination level.

Broiler litter will be handled according to commercial industry practice.

Iksin treated birds and litter will be destroyed to waste according to environmental standards. Untreated birds and positive control birds will be processed as broilers for human consumption.

Birds for Serial Monitoring

Five chicks from each replicate in each treatment group will be randomly selected for the collection of specific data throughout the study.

Specific Data

a) measurement of individual weight gain at 0, 14, 28 and 42 days (or slaughter);

b) Collection of cloacal swabs at 0, 14, 28, 42 days (or slaughter) for microbiological culture for the presence of Campylobacter and Salmonella;

c) Collection of breast muscle, liver and fat tissue at slaughter from Iksin treated birds+samples of broiler litter for measurement of Iksin residues;

d) Collection of breast muscle, liver and fat tissue+samples of broiler litter at 5 days post-withdrawal for measurement of Iksin residues;

e) Collection of faecal samples from Iksin treated birds for assay for Iksin;

f) Collection of caecal contents and caecal tissue to determine colonisation and invasion respectively with Campylobacter and Salimonella (mean viable count per treatment group).

General Data

a) Record of feed consumed between 0, 14, 28, 42 days (or slaughter) by weighback of feed in hoppers.

b) Observation for appearance, behaviour and general health and well-being.

c) Record of morbidity (deaths+unfit culls) and any gross pathology data.

Anlysis of Data

Weight gain and feed efficiency between treatment groups will be compared.

Pathogen colonisation rate in sentinel birds from all treatment groups will be determined by presence/absence of pathogens in cloacal samples.

The effect of Iksin treatment on Campylobacter colonisation (caecal contents) and invasion (caecal tissue) at slaughter will be compared to controls of the treatment group mean viable counts.

Expected Outcomes

The trial should show:

(i) whether there is a weight gain advantage over negative control birds at specific intervals and at time of processing;

(ii) how Iksin compares to a treatment regimen previously used in the industry;

(iii) whether Iksin reduces pathogen incidence (Salmonella and Campylobacter) in the gastrointestinal tract during rearing;

(iv) whether Iksin reduces pathogen loads or invasion by Campylobacter and salmonella into poultry tissues;

(v) whether there are any Iksin residues in muscle, fat and liver tissues at slaughter and after withdrawal;

(vi) the level of Iksin in broiler litter at slaughter and after withdrawal; and

(vii) whether there is any effect on growth rates of the use of clean versus contaminated litter.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

After entry into the U.S. national stage, and assurance of a U.S. filing date, the present document revises the claims from the PCT application by amending claims 1, 2, 5, 6, 8-16, 23, 24 and 25, and canceling claims 17-22. According to 37 C.F.R. § 1.121(c), after entry of the present amendment, the status of the claims in the case is as follows:

1. A method of promoting growth in a subject in need thereof, comprising administering a compound of formula I: 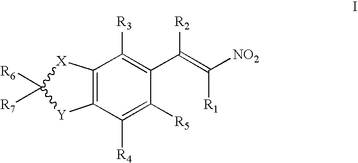
in which
X and Y are either the same or different and selected from a heteroatom; [symbol]
is a double or single bond depending on the heteroatoms X and Y;
R₁ to R₅ are either the same or different and selected from hydrogen or a non-deleterious substituent; and
R₆ and R₇ are either the same or different and selected from hydrogen and a non-deleterious substituent or one of R₆ and R₇ are absent when there is a double bond present,
or pharmaceutically or veterinarily acceptable salts or derivatives, pro-drugs, tautomers and/or isomers thereof,
to said subject in an amount effective to promote growth in said subject.

2. A method according to claim 1, in which the subject is an animal.

3. A method according to claim 2, in which the animal is an intensively farmed animal.

4. A method according to claim 3, in which the intensively farmed animal is a mammal, bird or fish.

5. A method according to claim 4, in which the mammal is a pig, cow or sheep.

6. A method according to claim 1, in which X and Y are either the same or different and selected from O and N.

7. A method according to claim 6, in which X and Y are both O.

8. A method according to claim 1, in which R₁ and R₂ are either the same or different and selected from hydrogen, hydroxy, halogen and optionally substituted C₁₋₆ alkyl.

9. A method according to claim 1, in which R₃ to R₅ are either the same or different and selected from hydrogen, hydroxy, halogen, nitro, C₁₋₆ alkoxy and optionally substituted C₁₋₆ alkyl.

10. A method according to claim 8 in which the halogen is chlorine or bromine.

11. A method according to claim 1, in which the compound of the formula I is in the form of an E isomer.

12. A method according to claim 11, in which X, Y, [symbol] , R₆ and R₇ are as defined in claim 1; R₁ and R₂ are either the same or different and selected from hydrogen, hydroxy, Cl, Br and C₁₋₄ alkyl; and R₃ to R₅ are either the same or different and selected from hydrogen, hydroxy, Cl, Br, nitro, C₁₋₄ alkoxy and C₁₋₄ alkyl.

13. A method according to claim 1, in which X and Y are O, R₁ is methyl and R₂ to R₇ are hydrogen (3,4-methylenedioxy-β-methyl-β-nitrostyrene) 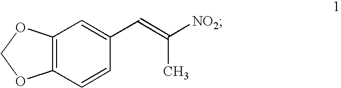
X and Y are O and R₁ to R₇ are hydrogen (3,4-methylenedioxy-β-nitrostyrene) 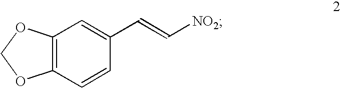
X is N, Y is NH, R₁ is methyl, R₂ to R₆ are hydrogen and R₇ is absent (benzimidazole-5-β-nitropropylene) 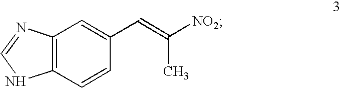
X is N, Y is NH, R₁ to R₅ are hydrogen, R₆ is methyl and R₇ is absent (2-methyl benzimidazole-5-β-nitroethylene) 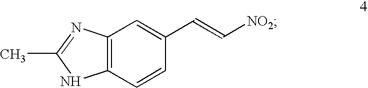
X is O, Y is N, R₁ to R₆ are hydrogen and R₇ is absent (benzoxazole-5-β-nitroethylene) 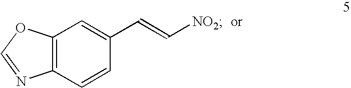
X is N, Y is O, R₁ is methyl, R₂ to R₅ are hydrogen, R₆ is methyl and R₇ is absent (2-methyl benzoxazole-5-β-nitropropylene)

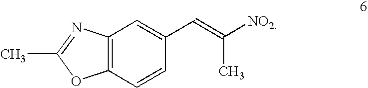

14. A method according to claim 1, in which the subject is a human.

15. A method according to claim 4, in which the bird is a chicken or turkey.

16. A method according to claim 9, in which the halogen is chlorine or bromine.

17.-22. (canceled)

23. A feed comprising the compound of formula I as defined in claim 1 and a carrier selected from alfalfa meal, soybean meal, cottonseed oil meal, linseed oil meal, corn meal, cane molasses, urea, bone meal, fish meal and corncob meal.

24. A method of promoting growth in a bird, comprising administering to said bird an amount of a compound effective to promote growth in said bird, wherein said compound is a compound of formula I: 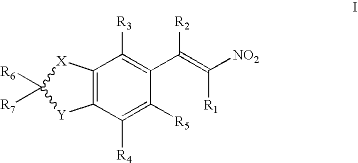
in which
X and Y are either the same or different and selected from a heteroatom; [symbol]
is a double or single bond depending on the heteroatoms X and Y;
R₁ to R₅ are either the same or different and selected from hydrogen or a non-deleterious substituent; and
R₆ and R₇ are either the same or different and selected from hydrogen and a non-deleterious substituent or one of R₆ and R₇ are absent when there is a double bond present,
or a pharmaceutically or veterinarily acceptable salt thereof.

25. Use of the compound of formula I as defined in any one of claims 1 to 13 as a growth promoting agent or nutritional supplement A method according to claim 24, in which the bird is a chicken or turkey.
